(19) United States
(12) Patent Application Publication
(10) Pub. No.: US 2026/0139175 A1
(43) Pub. Date: May 21, 2026

(54) USE OF AN AQUEOUS DEICING/ANTI-ICING COMPOSITION FOR REDUCING CARBON FIBRE DEGRADATION

(71) Applicant: PERSTORP AB, Perstorp (SE)

(72) Inventors: Marie WESTERBLAD, Hässleholm (SE); Erik HIMMER, Ballingslöv (SE)

(73) Assignee: PERSTORP AB, Perstorp (SE)

(21) Appl. No.: 19/121,269

(22) PCT Filed: Jul. 10, 2023
(86) PCT No.: PCT/SE2023/050725

§ 371 Date: Apr. 15, 2025

(30) Foreign Application Priority Data
Oct. 17, 2022 (SE) 2230333-3

(51) Int. Cl. C09K 3/18; B64F 5/23; C07C 53/06; C08B 11/02

(52) U.S. Cl. CPC C09K 3/185; B64F 5/23; C07C 53/06; C08B 11/02

(57) ABSTRACT

Disclosed is the use of an aqueous deicing/anti-icing composition for reducing carbon fibre degradation. Said composition comprises 40-60% by weight of at least one alkali metal carboxylate and 0.1-2.0% by weight of microfibrillated cellulose. The microfibrillated cellulose comprises at least one hydroxyalkyl cellulose. The balance to 100% by weight is achieved by water and said composition has a pH of 9-12. Disclosed is also a method for reducing carbon fibre degradation of an aqueous deicing/anti-icing composition in brakes. The method comprises applying the composition on an area selected from airfield runway, airfield taxiway, street, road, cycle way, footpath, sidewalk, bridge, backyard, parking area and sportsground. Disclosed is also the use of microfibrillated cellulose as a deicing/anti-icing composition additive for reducing carbon fibre degradation in airplane brakes.

### TECHNICAL FIELD

The present invention relates to the use of an aqueous deicing/anti-icing composition for reducing carbon fibre degradation. The present invention is also directed to a method for reducing carbon fibre degradation of an aqueous deicing/anti-icing composition in brakes, preferably airplane brakes. In a further aspect, the invention relates to the use of microfibrillated cellulose as a deicing/anti-icing composition additive for reducing carbon fibre degradation in airplane brakes.

### BACKGROUND SECTION

Removal (deicing) and prevention (anti-icing) of frost, ice or snow accumulation on, e.g., trafficked areas, such as airfield runways, airfield taxiways, streets, roads, cycle ways, footpaths, sidewalks, bridges, backyards, parking areas, sportsgrounds and similar areas is crucial to avoid substantial impairment in personal, traffic and transport safety. It is therefore known to apply compositions or compounds to such areas in order to melt snow and ice, thaw snow and ice or both. An essential requirement in this context is to achieve very rapid thawing.

In order to remove snow and ice both solid and liquid deicing and anti-icing compounds and compositions are used. Deicers and anti-icers commonly used in for instance trafficked areas include alkali metal, alkaline earth metal, amine and ammonium carboxylates and salts, such potassium, sodium, lithium, magnesium, calcium and ammonium formate, acetate, propionate, butyrate, isobutyrate, oxalate, malonate, succinate, glutarate, adipate, citrate, gluconate, benzoate, carbonate, bicarbonate, fluoride, chloride and bromide.

However, the materials from which above identified trafficked areas are made and materials from vehicles trafficking said areas are made, such as stone, bitumen and various metals, must not be damaged or otherwise destroyed by the deicing/anti-icing compositions.

One essential requirement for deicing and anti-icing compositions is thus to rule out corrosive actions on for instance metals, especially light metals for example used in parts of aircrafts and cars. An important test for deicing and anti-icing compositions that are used in areas such as aircraft runways is the immersion corrosion test disclosed in ASTM F 483-98, AMS 1431E and AMS 1435C which states that panels of dichromated magnesium alloy AZ31B-H26 must tolerate 5% by weight as well as 15% by weight of an aqueous deicing/anti-icing composition or a liquid deicing/anti-icing composition in undiluted form at 38° C. for 24 hours with a weight change of said panels being less than 0.2 mg/cm².

Many attempts have been made to address corrosion and hydrogen embrittlement of deicing/anti-icing compositions by the use of inhibitors. EP 0 375 214 discloses a liquid deicing composition consisting of 45-60% by weight of an alkali metal acetate/alkali metal formate, 0.1-0.4% by weight of an alkali metal phosphate and 0.2-0.6% by weight of an alkali metal nitrite. Yet, the inhibitor effect of alkali metal phosphate and alkali metal nitrite leaves room for improvement, particularly with regard to magnesium. A further disadvantage is the nitrite content. EP 3 478 788 discloses an aqueous deicing/anti-icing composition comprising 0.01-0.15% by weight of succinic acid, at least one alkali metal succinate or both and as active deicing/anti-icing component 40-60% by weight of at least one alkali metal carboxylate. This results in inhibition of corrosion, conductivity and hydrogen embrittlement.

Another general requirement for deicing and anti-icing compositions is their biodegradability as the compositions may potentially leak to water, and airfield operators are under significant public pressure regarding environmental safety and hence are pressed to use environmentally friendly compositions for that purpose. Deicing/anti-icing compositions comprising potassium formate as main ingredient, which is also used as a fertilizer component, are considered to be environmentally friendly and cost-effective.

### Technical Problem

While state of the art deicing/anti-icing composition may satisfy the general requirements identified above, there is still room for improvement with respect to the protection of materials getting into contact with said compositions.

This is in particular relevant when applying deicing/anti-icing compositions on airfield areas: Airplanes have brakes which are usually made from carbon fibres. Since these carbon fibre-based brakes are porous, deicing/anti-icing compositions may penetrate them. Alkali metals such as potassium within said compositions, however, may have a negative impact on the carbon fibres. When reducing the speed of an airplane, temperatures of 500° C. and more occur in the brakes. At these temperatures, the alkali metals catalyze the oxidation of the carbon fibres, which initiates their degradation. Accordingly, the lifetime of these carbon fibre brakes is drastically reduced, while their replacement is very costly.

Hence, there is a need to use a deicing/anti-icing composition which does not cause problems on materials, such as used in airplanes, by being aggressive on, e.g., the airframe.

### SUMMARY OF THE INVENTION

The present invention is based on the finding that the above problem of reduced carbon fibre degradation can be solved by the use of a deicing/anti-icing composition which inter alia includes microfibrillated cellulose as a component.

Thus, in a first aspect the present invention provides the use of an aqueous deicing/anti-icing composition for reducing carbon fibre degradation. The composition comprises 40-60% by weight of at least one alkali metal carboxylate and 0.1-2.0% by weight of microfibrillated cellulose which comprises at least one hydroxyalkyl cellulose. The balance to 100% by weight is water and said composition has a pH of 9-12.

In a second aspect, the invention provides a method for reducing carbon fibre degradation of an aqueous deicing/anti-icing composition in brakes. The method comprises applying said composition on an area selected from an airfield runway, airfield taxiway, street, road, cycle way, footpath, sidewalk, bridge, backyard, parking area and sportsground. The composition comprises 40-60% by weight of at least one alkali metal carboxylate and 0.1-2.0% by weight of microfibrillated cellulose which comprises at least one hydroxyalkyl cellulose. The balance to 100% by weight is water and said composition has a pH of 9-12.

In a third aspect, the invention provides the use of microfibrillated cellulose as a deicing/anti-icing composition additive for reducing carbon fibre degradation in airplane brakes.

### BRIEF DESCRIPTION OF THE FIGURE

FIG. 1: Mean oxidation weight losses of anti-oxidant coated carbon test coupons contaminated with the composition of the Embodiment Example or of the Comparative Example or without a deicing/anti-icing composition contamination (control group).

### DETAILED DESCRIPTION

We have quite surprisingly found that the use of microfibrillated cellulose in deicing/anti-icing compositions results in substantially reduced carbon fibre degradation and, hence, in improved lifetimes of airplane carbon brakes.

In the context of the present invention, the expression “deicing and/or anti-icing composition” is synonymous to “deicing/anti-icing composition” and refers to a composition which is either capable of removing (deicing) or preventing (anti-icing) frost, ice or snow accumulation or which is capable of both.

The expression “microfibrillated cellulose”, also known as nanocellulose, is a material consisting of cellulose fibres in nanoscale. Typical dimensions are 5-20 nm in width and up to 2000 nm in length. Microfibrillated cellulose acts as a gelling agent in water. Microfibrillated cellulose generally may be produced by mechanical treatment of cellulose with or without enzymatic or chemical pre-treatment. The process of fibrillation of cellulose fibers may for instance include the application of mechanical shearing.

In its first aspect, the present invention relates to the novel use of an aqueous deicing/anti-icing composition comprising 40-60% by weight of at least one alkali metal carboxylate and 0.1-2.0% by weight of microfibrillated cellulose for reducing carbon fibre degradation. The microfibrillated cellulose comprises at least one hydroxyalkyl cellulose, preferably hydroxyethyl cellulose. The balance to 100% by weight of the aqueous deicing/anti-icing composition is water. The pH of the composition according to the invention is 9-12. The pH value may be adjusted by further adding to the composition an alkali metal hydroxide, a carbonate or both.

In a preferred embodiment of the first aspect, the composition used for reducing carbon fibre degradation further comprises 0.1-0.5% by weight of benzoic acid, at least one alkali metal benzoate calculated as benzoic acid or both, 0.05-0.5% by weight of at least one alkali metal phosphate, 0.005-0.05% by weight of at least one alkali metal metasilicate, and optionally 0.01-0.15% by weight of succinic acid, at least one alkali metal succinate calculated as succinic acid or both.

In a more preferred embodiment of the first aspect of the present invention, the composition used for reducing carbon fibre degradation comprises 45-55% by weight of at least one alkali metal carboxylate, 0.1-2.0% by weight of microfibrillated cellulose, 0.2-0.4% by weight of benzoic acid, at least one alkali metal benzoate calculated as benzoic acid or both, 0.1-0.3% by weight of at least one alkali metal phosphate, 0.01-0.03% by weight of at least one alkali metal metasilicate, and 0.05-0.1% by weight of succinic acid, at least one alkali metal succinate calculated as succinic acid or both. The microfibrillated cellulose of this more preferred embodiment comprises at least one hydroxyalkyl cellulose, preferably hydroxyethyl cellulose and the pH of the composition is 10-11.

In preferred embodiments of the first aspect, said alkali metal carboxylate a) is a formate, an acetate or both. In a more preferred embodiment, the alkali metal carboxylate a) is a formate. In a more preferred embodiment, the alkali metal carboxylate a) is potassium formate.

In preferred embodiments of the first aspect, said at least one alkali metal metasilicate e) is at least one alkali metal silicate, said alkali metal being potassium, sodium or both.

In an even more preferred embodiment of the first aspect of the present invention, the composition used for reducing carbon fibre degradation comprises 45-55% by weight of potassium formate, potassium acetate, sodium formate or sodium acetate or a mixture comprising two or more thereof, 0.1-2.0% by weight of hydroxyethyl cellulose, 0.2-0.4% by weight of benzoic acid, potassium benzoate calculated as benzoic acid, sodium benzoate calculated as benzoic acid or a mixture comprising two or more thereof, 0.1-0.3% by weight of potassium phosphate, sodium phosphate or both, 0.01-0.03% by weight of potassium metasilicate, sodium metasilicate, an anhydrous species, a hydrate or a mixture comprising two or more thereof, and 0.05-0.1% by weight of succinic acid, potassium succinate calculated as succinic acid, sodium succinate calculated as succinic acid or a mixture comprising two or more thereof.

The composition used for reducing carbon fibre degradation according to the first aspect of the present invention may in its various embodiments additionally comprise at least one oxygen scavenger g) in an amount of 0.01-0.5% by weight, said at least one oxygen scavenger being particularly preferred potassium hydrogen sulphite, sodium hydrogen sulphite or both.

The use according to any of above embodiments of the first aspect of the present invention may relate to deicing/anti-icing of an area selected from airfield runway, airfield taxiway, street, road, cycle way, footpath, sidewalk, bridge, backyard, parking area and sportsground, more preferably to deicing/anti-icing of an airfield runway.

In its second aspect, the present invention relates to a method for reducing carbon fibre degradation in brakes, preferably airplane brakes, by an aqueous deicing/anti-icing composition. The method comprises applying said composition on an area selected from airfield runway, airfield taxiway, street, road, cycle way, footpath, sidewalk, bridge, backyard, parking area and sportsground, preferably an airfield runway. The composition comprises 40-60% by weight of at least one alkali metal carboxylate, preferably alkali metal formate, alkali metal acetate or both and 0.1-2.0% by weight of microfibrillated cellulose. The microfibrillated cellulose comprises at least one hydroxyalkyl cellulose, preferably hydroxyethyl cellulose. The balance to 100% by weight of the aqueous deicing/anti-icing composition is water. The pH of the composition according to the second aspect of the invention is 9-12 and may be adjusted as described above for the first aspect of the present invention.

In a preferred embodiment of the second aspect, the composition of the method for reducing carbon fibre degradation further comprises 0.1-0.5% by weight of benzoic acid, at least one alkali metal benzoate calculated as benzoic acid or both, 0.05-0.5% by weight of at least one alkali metal phosphate, 0.005-0.05% by weight of at least one alkali metal metasilicate being preferably at least one alkali metal silicate and said alkali metal preferably being potassium, sodium or both, and optionally 0.01-0.15% by weight of succinic acid, at least one alkali metal succinate calculated as succinic acid or both being preferably potassium succinate, sodium succinate or both.

In a more preferred embodiment of the second aspect of the present invention, the composition of the method for reducing carbon fibre degradation comprises 45-55% by weight of at least one alkali metal carboxylate. Furthermore, the composition comprises 0.1-2.0% by weight of microfibrillated cellulose, said microfibrillated cellulose being at least one hydroxyalkyl cellulose, 0.2-0.4% by weight of benzoic acid, alkali metal benzoate calculated as benzoic acid or both, 0.1-0.3% by weight of at least one alkali metal phosphate, 0.01-0.03% by weight of at least one alkali metal metasilicate, and 0.05-0.1% by weight of succinic acid, at least one alkali metal succinate calculated as succinic acid or both. The pH of the composition applied according to the second aspect is 10-11.

In an even more preferred embodiment of the second aspect of the present invention, the composition of the method for reducing carbon fibre degradation comprises 45-55% by weight of alkali metal formate, alkali metal acetate or both, preferably alkali metal formate and more preferably potassium formate. Furthermore, the composition comprises 0.1-2.0% by weight of hydroxyethyl cellulose, 0.2-0.4% by weight of benzoic acid, potassium benzoate calculated as benzoic acid, sodium benzoate calculated as benzoic acid or a mixture comprising two or more thereof, 0.1-0.3% by weight of potassium phosphate, sodium phosphate or both, 0.01-0.03% by weight of at least one alkali metal silicate and said alkali metal preferably being potassium, sodium or both, and 0.05-0.1% by weight of succinic acid, potassium succinate calculated as succinic acid, sodium succinate calculated as succinic acid or a mixture comprising two or more thereof. The pH of the composition applied according to the second aspect is 10-11.

The composition of the method for reducing carbon fibre degradation according to the second aspect of the present invention may in its various embodiments additionally comprise at least one oxygen scavenger g) in an amount of 0.01-0.5% by weight, said at least one oxygen scavenger being particularly preferably potassium hydrogen sulphite, sodium hydrogen sulphite or both.

In its third aspect, the present invention relates to the use of microfibrillated cellulose as a deicing/anti-icing composition additive for reducing carbon fibre degradation in airplane brakes. Said additive may be added for instance to deicing/anti-icing composition that are commonly known or commercially available in order to substantially reduced carbon fibre degradation and thus improve lifetimes of airplane carbon brakes.

### EXPERIMENTAL SECTION

The present invention as defined by the claims and its effect is further exemplified by the following Embodiment Example and the Comparative Example deicing/anti-icing compositions and the results of a catalytic carbon brake disk oxidation test performed in accordance with SAE AIR5567A.

### Embodiment Example

An aqueous deicing/anti-icing composition according to the invention was prepared by mixing and dissolving:

| | | |
|---|---|---|
| 50.0% | by weight | potassium formate, |
| 0.50% | by weight | microfibrillated cellulose (Exilva ® |
| | | Piano, by Borregard, Norway), |
| 0.30% | by weight | sodium benzoate, |
| 0.15% | by weight | potassium phosphate, |
| 0.015% | by weight | sodium metasilicate pentahydrate, and |
| 0.075% | by weight | succinic acid. |

Water was added to the balance of 100% by weight and the pH was adjusted to 10.5±0.2 by the addition of potassium hydroxide.

### Comparative Example

The Embodiment Example was repeated with the difference that no microfibrillated cellulose was added.

### Catalytic Carbon Brake Disk Oxidation Test

The Embodiment Example and the Comparative Example deicing/anti-icing compositions were tested in accordance with the SAE AIR5567A “Test Method for Catalytic Carbon Brake Disk Oxidation” (revision A, June 2010) as per the latest revision of the SAE AMS1431 (“Solid Runway Deicing/Anti-Icing Product”, revision E, October 2018) and SAE AMS1435 (“Fluid, Generic, Deicing/Anti-Icing Runways and Taxyways”, revision C, June 2012).

In a first step, CARBENIX® 4000 (C4000® by Honeywell Aerospace, USA) test coupons were treated with a generic anti-oxidant (AO) coating. CARBENIX® 4000 is a carbon/carbon composite brake material that is in accordance with the SAE AIR5567A requirements.

In a second step, the deicing/anti-icing compositions were diluted to a concentration of 25% w/w. Next, the AO coated coupons were immersed in the diluted compositions of the Embodiment Example or the Comparative Example, respectively, for 10 minutes. The coupons were dried at 150° C. for 2 hours, transferred to a desiccator and cooled for a minimum of 30 minutes.

In a third step, the deicing/anti-icing composition-contaminated AO coated coupons were submitted to an oxidation test and heated in a still air muffle furnace at 550° C. for 24 hours. After cooling the coupons, the mean oxidation weight losses (%) were calculated in line with SAE AIR5567A.

As a control group, AO coated coupons that were not immersed in deicing/anti-icing compositions, i.e. only subjected to the first step, were directly submitted to the oxidation tests according to the third step described above.

The test described above was performed eight times each for the Embodiment Example, the Comparative Example and a control group, and a mean value of the results was calculated.

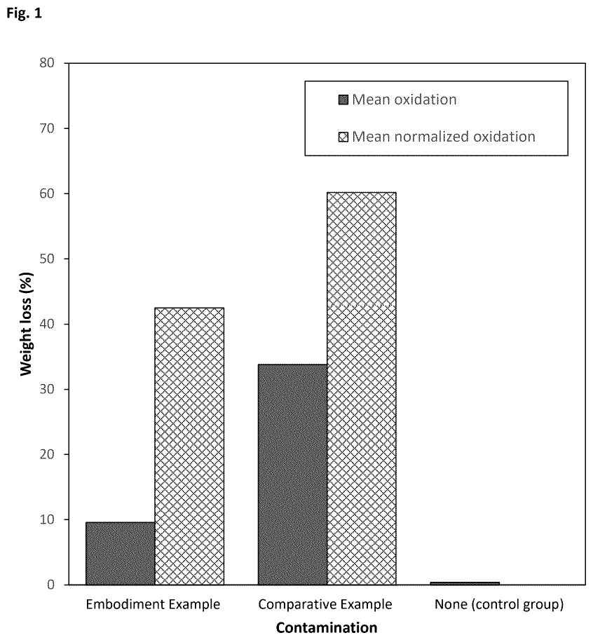

Table 1 summarizes the catalytic carbon brake disk oxidation test results. FIG. 1 illustrates the calculated mean oxidation weight losses.

TABLE 1
Summary of the catalytic carbon brake disk oxidation test results.

| | Mean oxidation | Mean normalized |
|---|---|---|
| Contaminant of AO coated | weight loss | oxidation weight loss |
| carbon coupons | (%) | (%) |
| Embodiment Example | 9.6 ± 2.9 | 42.5 ± 8.2 |
| Comparative Example | 33.8 ± 8.2 | 60.2 ± 13.2 |
| None (control group) | 0.4 ± 0.1 | n.a. |

In summary, it is shown that the use of an aqueous deicing and/or anti-icing composition in accordance with the invention is efficient for reducing carbon fibre degradation as it leads to significant reduced oxidation weight loss in carbon brakes compared to, e.g., compositions not including microfibrillated celluose. In light thereof, the present deicing and/or anti-icing composition can be suitably used on trafficked areas such as airfield runways or taxiways without having a negative impact such as carbon fibre degradation in materials being in contact therewith, such as airplane brakes.

1-14. (canceled)

15. A method for de-icing an area or preventing ice formation on the area, while minimizing degradation of carbon fibres in a brake system, the composition comprising:
(a) 40 to 50 wt. % of one or more alkali metal carboxylates; and
(b) 0.1 to 2.0 wt. % of microfibrillated cellulose, wherein the microfibrillated cellulose comprises at least one hydroxyalkyl cellulose;
wherein the composition has a pH of 9 to 12, and all weight percentages are based on a total weight of the de-icing and/or anti-icing composition.

16. The method of claim 15, wherein the aqueous de-icing and/or anti-icing composition further comprises:
(c) 0.1 to 0.5 wt. % of benzoic acid and/or one or more alkali metal benzoates, calculated as benzoic acid;
(d) 0.05 to 0.5 wt. % of one or more alkali metal phosphates;
(e) 0.005 to 0.05 wt. % of one or more alkali metal metasilicates; and
(f) optionally, 0.01 to 0.15 wt. % of succinic acid and/or one or more alkali metal succinates, calculated as succinic acid.

17. The method of claim 16, wherein the aqueous de-icing and/or anti-icing composition comprises:
(a) 45 to 55 wt. % of one or more alkali metal carboxylates;
(b) 0.1 to 2.0 wt. % of microfibrillated cellulose, wherein the microfibrillated cellulose comprises a hydroxyalkyl cellulose;
(c) 0.2 to 0.4 wt. % of benzoic acid and/or one or more alkali metal benzoates, calculated as benzoic acid;
(d) 0.1 to 0.3 wt. % of one or more alkali metal phosphates;
(e) 0.01 to 0.03 wt. % of one or more alkali metal metasilicates; and
(f) 0.05 to 0.1 wt. % of succinic acid and/or one or more alkali metal succinates, calculated as succinic acid;
wherein the composition has a pH of 10 to 11.

18. The method of claim 17, wherein the hydroxyalkyl cellulose is hydroxyethyl cellulose.

19. The method of claim 18, wherein at least one of the one or more alkali metal carboxylates is a formate or an acetate.

20. The method of claim 19, wherein the at least one alkali metal carboxylate is the formate.

21. The method of claim 20, wherein the formate is potassium formate.

22. The method of claim 16, wherein at least one of the one or more alkali metal metasilicates is an alkali metal silicate.

23. The method of claim 22, wherein the alkali metal silicate is potassium silicate or sodium silicate.

24. The method of claim 16, wherein the aqueous de-icing and/or anti-icing composition comprises:
(a) 45 to 55 wt. % of potassium formate, sodium formate, potassium acetate, sodium acetate, or combinations thereof;
(b) 0.1 to 2.0 wt. % of hydroxyethyl cellulose;
(c) 0.2 to 0.4 wt. % of benzoic acid, potassium benzoate, sodium benzoate, or combinations thereof, calculated as benzoic acid;
(d) 0.1 to 0.3 wt. % of potassium phosphate, sodium phosphate, or a combination thereof;
(e) 0.01 to 0.03 wt. % of potassium metasilicate, sodium metasilicate, an anhydrous species thereof, a hydrate thereof, or combinations thereof; and
(f) 0.05 to 0.1 wt. % of succinic acid, potassium succinate, sodium succinate, or combinations thereof, calculated as succinic acid.

25. The method of claim 24, wherein the aqueous de-icing and/or anti-icing composition further comprises:
(g) 0.01 to 0.5 wt. % of one or more oxygen scavengers.

26. The method of claim 25, wherein at least one of the one or more oxygen scavengers is potassium sulphite or sodium hydrogen sulphite.

27. The method of claim 15, wherein the area is selected from an airfield runway, an airfield taxiway, a street, a road, a cycle way, a footpath, a sidewalk, a bridge, a backyard, a parking area, and a sportsground.

28. The method of claim 27, wherein the area is the airfield runway.

29. The method of claim 15, wherein the method minimizes degradation of carbon fibres in a brake system exposed to the aqueous de-icing and/or anti-icing composition.

30. The method of claim 29, wherein the area is an airfield runway, an airfield taxiway, a street, a road, a cycle way, a footpath, a sidewalk, a bridge, a backyard, a parking area, or a sportsground.

31. The method of claim 29, wherein the brake system is part of an airplane.
